(19) United States
(12) Patent Application Publication
(10) Pub. No.: US 2009/0082664 A1
(43) Pub. Date: Mar. 26, 2009

(54) Endorectal Coil for Magnetic Resonance Tomograph

(75) Inventor: Harry Schilling, Eichstaett (DE)

Correspondence Address: DAFFER MCDANIEL LLP, P.O. BOX 684908, AUSTIN, TX, 78768, US

(73) Assignee: SCHLEIFRING UND APPARATEBAU GMBH, Fuerstenfeldbruck (DE)

(21) Appl. No.: 12/233,915

(22) Filed: Sep. 19, 2008

(30) Foreign Application Priority Data
Sep. 26, 2007 (DE) 102007046169.2

(51) Int. Cl. A61B 5/055

(52) U.S. Cl. 600/422

(57) ABSTRACT

An endoscopic RF coil arrangement for magnetic resonance imagers is provided which include a handle with a shaft and a coil with a balloon arranged distally. An inner cover is further provided which is pulled over the shaft and over the coil with balloon. An outer cover is provided over the inner cover. The outer cover comprises a formed part for a balloon on the distal side over the coil in which a liquid and/or a gas can be introduced.

### PRIORITY CLAIM

This application claims priority from German Patent Application 10 2007 046 169.2 filed on Sep. 26, 2007.

### BACKGROUND OF THE INVENTION

1. Field of the Invention

The invention relates to magnetic resonance imaging and especially to medical appliances for examining human and animal bodies.

2. Description of the Related Art

Magnetic Resonance Imaging (MRI) is an imaging process which is based on the physical phenomenon of nuclear magnetic resonance (NMR). The terms NMR and MRI are often used interchangeably to reference the same process scheme. In such processes, the object to be examined is subjected to a strong magnetic field. As a consequence of this, the nuclear spins of individual atoms will align, which before were statistically distributed. As a result of external stimulation with high-frequency energy, a measurable oscillation is excited. In order to enable spatial localization, magnetic fields are generated by gradient coils in the three spatial axes. Transmitting coils are provided for transmitting the high-frequency excitation energy. The excited oscillations are received by receiving coils. Transmitting and receiving coils are often also combined with each other. These various types of coils are also designated herein as RF coils since they are used for injecting and tapping the high-frequency signals.

As a result of the non-invasive imaging processes of MRI, slice images at random axes can be taken of the human or animal body.

Exemplary transmitting and receiving coils are disclosed in U.S. Pat. No. 4,887,039. Several parallel conductors are provided in a cylindrical support, which conductors are connected with each other by coupling capacitive elements. Feeding occurs by means of symmetrical lines or coaxial cables. So-called phased-array arrangements as are shown in FIG. 4 of this patent are used in order to achieve high resolutions. In this case, several independent coils are connected with independent receiver inputs for separate evaluation of the signals. Other coils are used for endoscopic examinations. An example of such a coil is disclosed in U.S. Pat. No. 6,606,513. Numerous antenna arrangements are provided, but it is necessary for performing a biopsy to remove the coil through a trocar or an outer sleeve from the body and to introduce a biopsy needle or another instrument instead. This leads to problems in the exact localization of a position previously chosen by MRI since the instrument can be displaced by the change of instrument. An improvement is offered by U.S. Pat. No. 6,898,454. This document also discloses an MRI coil. An additional balloon at the end of the instrument helps fix it. Furthermore, the coil comprises a duct in its interior, a duct through which a further instrument can be guided.

The disadvantageous aspect in coils known from the state of the art is that they can usually only be used for one surgery. The sensitive components in the interior of such coils cannot withstand any thermal sterilization. As a result of the complex arrangement with a large number of gaps and transition points, gas sterilization is also not possible.

### BRIEF SUMMARY OF THE INVENTION

The following description of various embodiments of coil arrangements for a magnetic resonance tomograph and embodiments of endoscopic instruments is not to be construed in any way as limiting the subject matter of the appended claims.

An embodiment of an endoscopic RF coil arrangement for a magnetic resonance tomograph includes a handle attached to a shaft, a coil arranged distally to the shaft, an inner cover which is pulled over the shaft and the coil, and an outer cover enclosing the inner cover. The outer cover includes a formed part for a balloon in its distal region for filling the volume between the outer cover and the inner cover with a liquid and/or gas to change the balloon in its size and/or to stiffen the balloon.

A further embodiment of an endoscopic RF coil arrangement for a magnetic resonance tomograph includes a handle attached to a shaft, a coil arranged distally to the shaft, an outer cover which is pulled over the shaft and the coil, and a support for an additional instrument, which is attached outside of the outer cover and penetrates the coil.

An embodiment of an endoscopic instrument includes a handle attached to a shaft, an instrument arranged distally to the shaft, an outer cover which encloses the shaft and a head of the instrument, and at least one support being provided for receiving an additional instrument which is also completely enclosed by the outer cover.

### BRIEF DESCRIPTION OF THE DRAWINGS

The invention is described below in an exemplary manner without any limitation to the inventive idea on the basis of embodiments by reference to the drawings, wherein:

FIG. 1 shows a coil arrangement for a magnetic resonance imager;

FIG. 2 shows the inner configuration of the coil arrangement depicted in FIG. 2;

FIG. 3 shows an inner cover for the inner configuration of the coil arrangement depicted in FIG. 2;

FIG. 4 shows an outer cover for the coil arrangement depicted in FIG. 1;

FIG. 5 shows a sectional view of the coil arrangement depicted in FIG. 1 from below;

FIG. 6 shows a sectional side view of the coil arrangement depicted in FIG. 1;

FIG. 7 shows a view of the coil arrangement depicted in FIG. 1 from the distal side;

FIG. 8 shows a schematic general view of an apparatus for magnetic resonance imaging;

FIG. 9 shows a sectional view of a coil arrangement with an additional instrument attached on the outside;

FIG. 10 shows an embodiment of the coil arrangement depicted in FIG. 9 in a top view;

FIG. 11 shows an alternative embodiment of the coil arrangement depicted in FIG. 9 in a top view;

FIG. 12 shows a coil arrangement with a straight coil in a side view;

FIG. 13 shows the coil arrangement depicted in FIG. 12 in a top view;

FIG. 14 shows the coil arrangement depicted in FIG. 12 in a perspective view.

### DETAILED DESCRIPTION OF PREFERRED EMBODIMENTS

An endoscopic RF coil arrangement for magnetic resonance imagers is described herein which includes a handle at the proximal end, a shaft in connection with the handle, a coil arranged distally on the shaft with a balloon. The shaft includes an inside tube, at the distal end of which there is a coupling for receiving a plug of the coil. The coil can thus be connected with the shaft or be detached from the same. It is advantageous for example for cleaning, sterilizing or even exchanging the coil. The coil with the balloon, shaft and portions of the handle can be covered with an inner cover. The inner cover includes a formed element for the coil. Furthermore, an outer cover is provided which completely encloses the inner cover and includes a formed part at its distal region for a balloon. A liquid and/or a gas can be introduced into the volume between the outer cover and the inner cover, especially into the formed part for the balloon, in order to inflate the balloon. The liquid preferably has the same or similar electric, especially dielectric, properties as the surrounding tissue. It preferably includes an emulsion with perfluorooctyl bromide. The balloon can thus be changed in its size and stiffness.

As a result of the arrangement with an inner cover and an outer cover, the arrangement can be mounted, dismounted and thus also cleaned and maintained with ease. After an application, merely the outer cover typically needs to be changed. An outer cover with an adjusted balloon size can preferably be applied according to the next case of operation. A large balloon size can be chosen in case of large body cavities or large patients. Small balloon sizes can be used in opposite cases.

As a result of the simple exchangeability of the inner cover and especially the outer cover, sterilization of the entire apparatus is no longer required. Sensitive electronic components can thus also be integrated in the arrangement. A preamplifier or even an electronic switch is preferably also integrated in the coil or the printed circuit board of this coil. Alternatively, these components can also be housed in other areas of the instrument, e.g. in the handle.

A further advantageous embodiment provides that at least one connection is provided for introducing a liquid and/or a gas into the volume between the outer cover and the inner cover. The connection can advantageously be combined with a tap or a valve.

In a further advantageous embodiment, at least one connection is provided for evacuating the volume between the inner cover and the parts enclosed by the inner cover. These parts are especially the inside tube, the coupling, the plug and the coil.

In an advantageous embodiment, the inner cover is sealed with a seal against a flaring on the coil arrangement. Fixing by means of a fastening element occurs advantageously. The flaring is advantageously attached to the proximal end of the shaft, at the transition point to the handle.

In a further advantageous embodiment, the outer cover is sealed with a seal against a flaring on the coil arrangement. It is especially advantageous when the fixing occurs by means of a fastening element. The flaring is advantageously attached to the proximal end of the shaft at the transition point to the handle, so that the outer cover completely encloses the inner cover.

According to a further embodiment, an endoscopic RF coil arrangement for magnetic resonance imagers includes a handle with a shaft and a distally arranged coil. Moreover, an outer cover is provided which is pulled over the shaft and over the coil. A support for an additional instrument is arranged outside of the outer cover. The inserted additional instrument can penetrate the coil. For this purpose, the coil comprises an opening in its interior. The coil is thus arranged or wound about the additional instrument. Prior to using the arrangement, an outer cover is pulled over the coil and the shaft. At least one connection for evacuating or filling the cavity between the outer cover and the instrument can be provided. The coil can also be arranged in a fork-like manner.

In a further advantageous embodiment, a plug-and-socket connection is provided, which includes a coupling and a plug for electric connection of the coil to the connecting cable and for mechanical fastening to the inside tube. The coupling is advantageously connected with the inside tube. The plug can alternatively also be connected to the inside tube and the coupling to the coil.

It is further advantageous when the antenna includes a printed circuit board. If the antenna is to be elastic or flexible, it can also include a flexible printed circuit board. The antenna is advantageously a receiving coil. It can also be any other kind of antenna according to the state of the art.

It is also advantageous when different coils are provided. An adjustment to different examination tasks can be provided by an exchange of the coils. A matching set of an inner cover and an outer cover is further preferably provided for different coil sizes.

An actuating unit is provided in a further embodiment which can influence the position of the coil arrangement or the entire instrument. The actuating unit can be triggered by control signals of a computer system for example. Adjustments can thus be made automatically or at least in a remote-controlled way without the staff having to be present close to the magnetic resonance imager.

In a further embodiment, at least one support is provided for receiving an additional instrument. Such an additional instrument can be a biopsy needle for example. The support is completely enclosed by the outer cover. The additional instrument is thus located outside of the outer cover. Prior to use of the instrument, it needs to be covered with a new outer cover. The outer cover can be arranged as a disposable article. The additional instrument needs to be sterilized prior to insertion. A disposable instrument can alternatively also be used. As a result of this combination it is not necessary to sterilize the complete instrument which contains a sensitive coil for example. The additional instrument can be clamped in the support for example. The support can be provided with a flexible or rigid configuration. In the case of a rigid support, the material enclosing the outer cover can supply the necessary elasticity. The outer cover is thus advantageously produced from an elastic, preferably rubber-like, material. It is especially advantageous when the additional support is arranged like a clamp, e.g. with two approximately parallel legs or legs which at least partly enclose a round additional instrument. Further supports are advantageously provided in order to guide the additional instrument in a better way parallel to the shaft of the instrument. Furthermore, at least one guide means can further be provided in order to improve the stability of the additional instrument against the instrument.

The embodiments described herein are not limited to a coil arrangement for magnetic resonance imagers. They can be applied to general endoscopic instruments. Therefore, an endoscopic instrument is arranged according to another aspect of the invention in such a way that an outer cover is provided which encloses the shaft and the head of the instrument. Moreover, at least one support for receiving an additional instrument is provided which is completely enclosed by the outer cover. In accordance with the invention, all previously described features and variants of embodiments can be combined.

It is especially advantageous when the outer cover comprises a formed part for a balloon at its distal end and a liquid and/or gas can be introduced into the volume between the outer cover and the head of the instrument in order to change the balloon in its size and/or to stiffen the same.

It is further advantageous when an inner cover is provided which encloses at least the head of the instrument. Effective sealing for the liquid and/or the gas in the outer cover can thus be achieved.

Moreover, the various features of the embodiments as described herein can be combined with each other. It is the object of the invention to provide a coil arrangement which allows a biopsy at a point previously determined precisely by MRI. Moreover, the instrument shall be useful for several uses with a low amount of effort. Finally, the instrument shall be adaptable to a large number of different tasks.

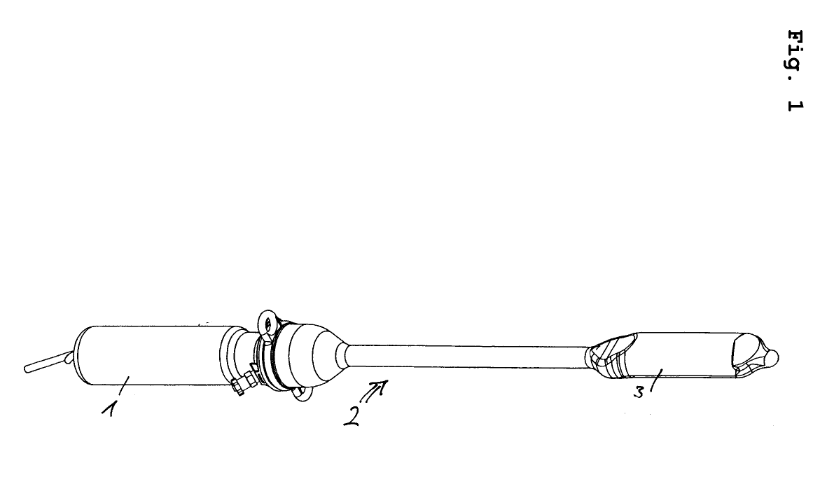

FIG. 1 shows a general form of an endoscopic RF coil arrangement. The endoscopic RF coil arrangement for magnetic resonance imagers comprises a handle 1 on the proximal side which is connected with a shaft 2 which comprises at its distal end a coil and a balloon 3.

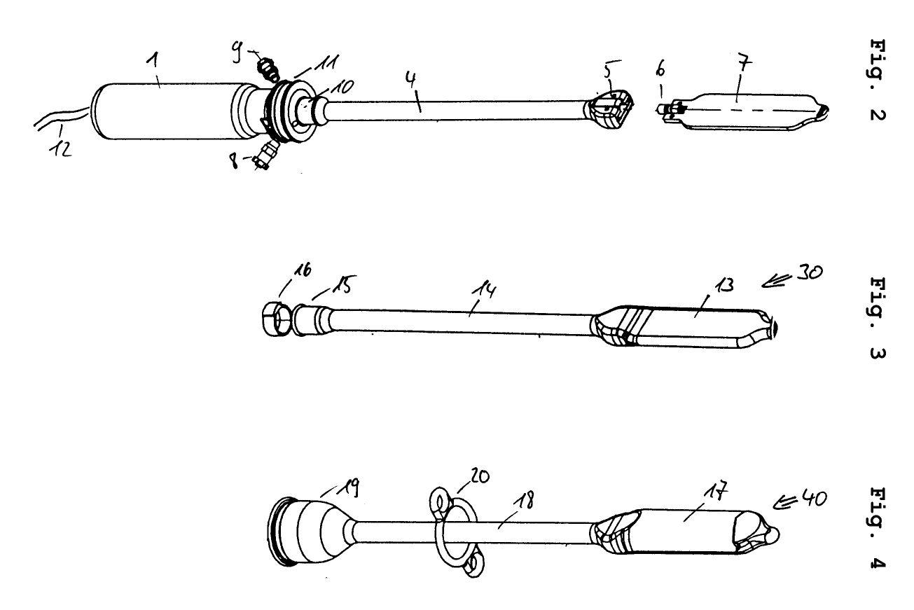

FIG. 2 shows the inner configuration of a coil arrangement. The proximal handle 1 is used for handling and guiding the entire arrangement. An inside tube 4 is attached to the same. The inside tube 4 forms the shaft of the instrument. A coil 7 is provided at the distal end of the shaft. The coil is connected mechanically with the inside tube and electrically with the connecting cable 12 by means of a plug-and-socket connection consisting of the coupling 5 and the plug 6. In the region between the handle 1 and the inside tube 4, a first connection 8 is provided for evacuating the volume between an inner cover 30 (depicted in FIG. 3) and the parts enclosed by the inner cover. Furthermore, a second connection 9 is provided for filling the balloon. Finally, a flaring 11 for an outer cover and a flaring 10 for the inner cover are provided in the direction of the inside tube 4. The flaring 11 for the outer cover is attached in such a way that the outer cover completely encloses the inner cover.

FIG. 3 shows an inner cover 30. It comprises a formed part 13 for receiving the coil 7 of FIG. 2. Adjacent thereto there is a cylindrical region 14 as a sleeve for the shaft which receives the inside tube 4 of FIG. 2. A seal 15 is provided at the proximal end which is slid over the flaring 10 of FIG. 2 for the inner cover. A fastening element 16 is applied over the inner cover for fixing purposes.

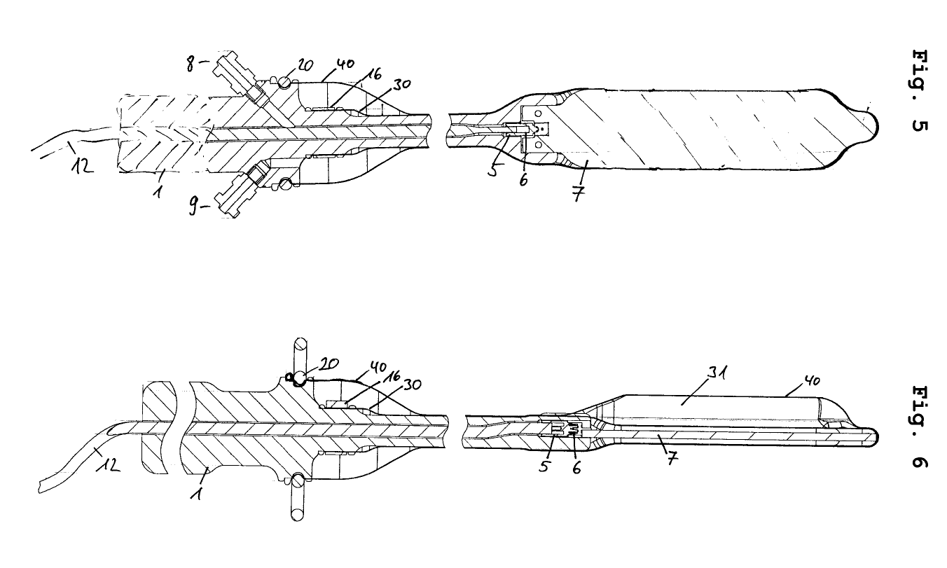

FIG. 4 shows an outer cover 40. It comprises a formed part 17 for a balloon. Adjacent thereto there is a cylindrical region 18 as a cover for the shaft. A seal 19 is provided at the proximal end which is slid over the flaring 11 of FIG. 2 for the outer cover. A fastening element 20 is applied over the inner cover for fastening purposes. The outer cover is preferably arranged in such a way that substantially only the formed part 17 for the balloon will fill or expand during filling with air and/or gas and the remaining regions, especially the shaft cover 18 and the seal 19, remain in a non-expanded state. This can be realized by a stiffer or thicker material or by reinforcing elements in the region of the shaft. FIG. 5 shows a sectional view of the endoscopic RF coil arrangement depicted in FIG. 1 from below.

FIG. 6 shows a lateral sectional view of the endoscopic RF coil arrangement depicted in FIG. 1. The filled balloon 31 is easy to recognize in this illustration.

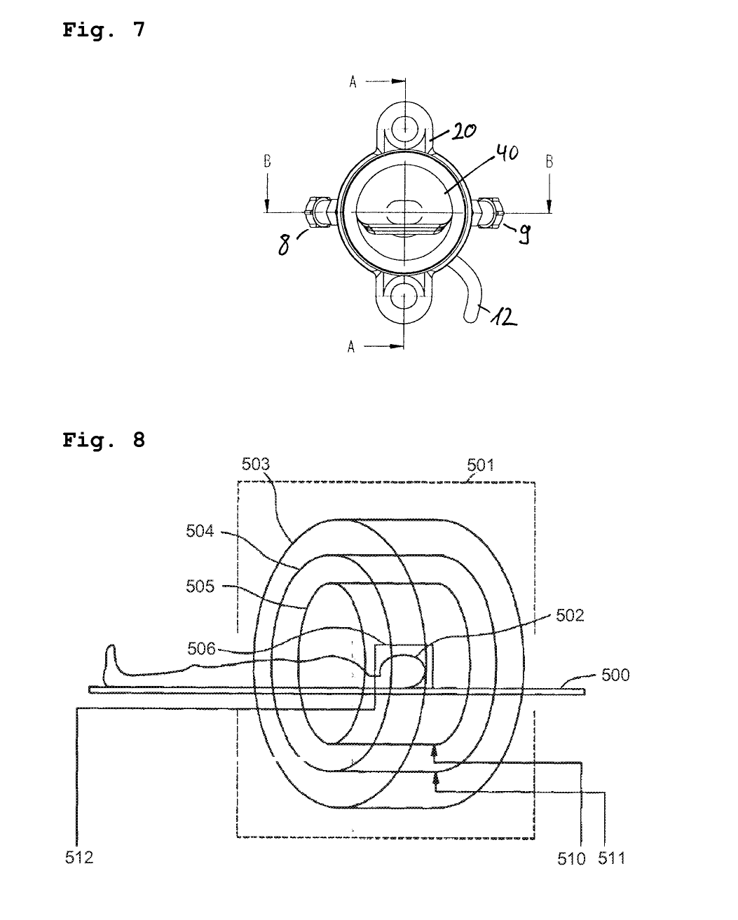

FIG. 7 shows in a view of the endoscopic RF coil arrangement depicted in FIG. 1 from the distal side. Sectional view A-A is shown in FIG. 6. FIG. 5 shows the sectional view B-B.

FIG. 8 schematically shows an apparatus for magnetic resonance imaging in general form. The patient 502 rests on a bed 500 in the magnetic system 501. Animals or any other object could be examined instead of a patient. A main magnet 503 is used for generating the static main magnetic field. Temporally and locally variable magnetic fields are generated by means of the gradient coils 504 for position fixing. These are triggered by gradient signals 511. The high-frequency field for exciting the nuclear magnetic resonances is introduced with the help of the transmission signal 510 by the transmitting coils 505 into the object to be examined. The detection of the measuring signals 512 occurs by means of the receiving coils 506. Transmitting coils and receiving coils can optionally be combined with each other spatially. Similarly, the same coil arrangement can be used at first for signal transmission and later for signal receiving. The receiving coils 506 can also be arranged in the outside area of transmitting coils 505. Similarly, the transmitting coils 505 can be arranged close to the objects to be examined, like the receiving coils 506 shown here.

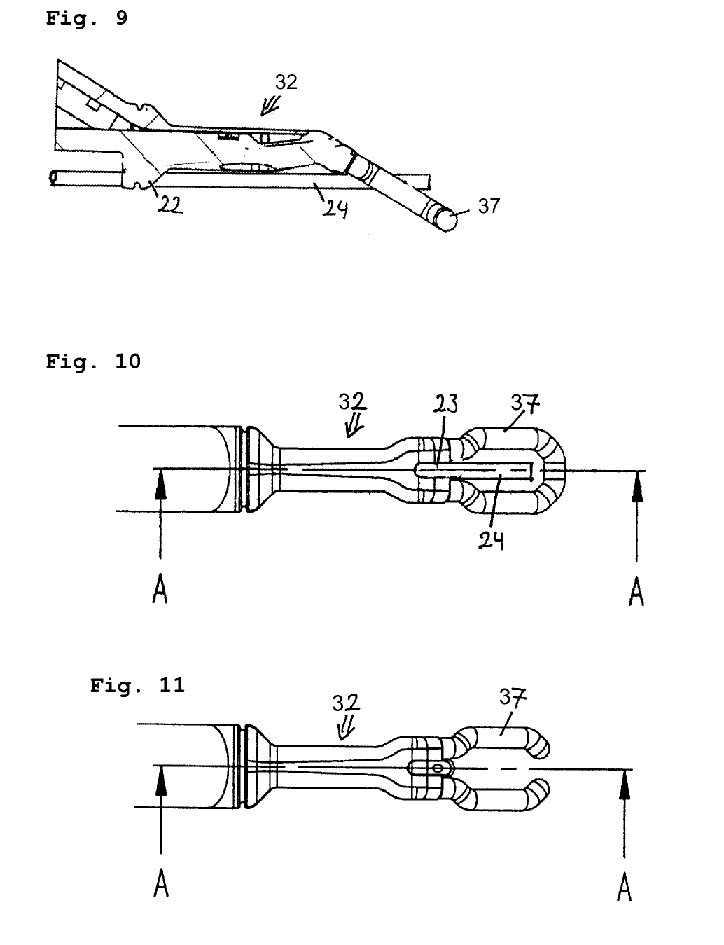

FIG. 9 shows a coil arrangement with an externally attached additional instrument in a sectional view. The coil arrangement stands here for any desired surgical instrument. The relevant fact is that the complete instrument, including the shaft 32 and the distal end which comprises a coil 37 in this case, is enclosed by an outer cover. An additional instrument 24 like a biopsy needle for example is attached to the outside on the coil arrangement. A support 22 and a guide means 23 (depicted in FIG. 10) are provided in this case for fastening the additional instrument. With this embodiment, all previously described features such as inner cover, outer cover and balloon can be combined. They are not shown completely for reasons of better clarity of the illustration.

FIG. 10 shows an embodiment of the coil arrangement depicted in FIG. 9 in a top view. The previously shown sectional view of FIG. 9 corresponds to the sectional designation A-A.

FIG. 11 shows an alternative embodiment of the coil arrangement depicted in FIG. 9 in the top view. The previously shown sectional view of FIG. 9 corresponds to the sectional designation A-A. The coil arrangement is open in this case, so that the additional instrument can be inserted more easily.

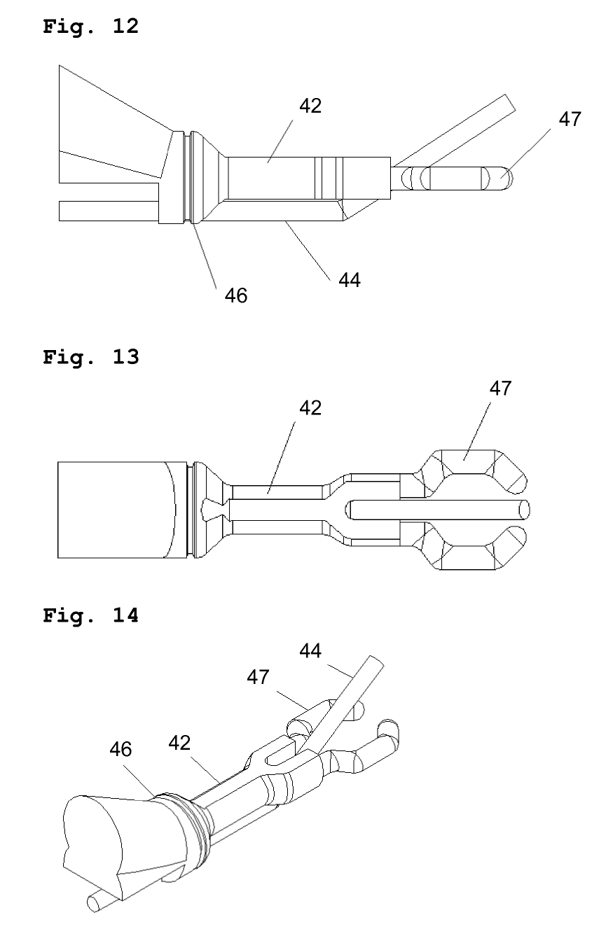

FIG. 12 shows a further coil arrangement in which the coil 47 is situated on the same axis as the shaft 42, in contrast to the arrangement shown in FIG. 9. The coil is not bent relative to the shaft. On the other hand, the additional instrument 44 such as a biopsy needle is bent and penetrates the coil 47. Similarly, in this straight configuration it is possible to realize a closed coil, as is shown in FIG. 10 for example. A support 46 is provided in this case for fastening the additional instrument.

FIG. 13 shows the coil arrangement of FIG. 12 in a top view.

FIG. 14 shows the coil arrangement of FIG. 12 in a perspective view.

1. An endoscopic RF coil arrangement for a magnetic resonance tomograph, comprising:
a handle attached to a shaft;
a coil arranged distally to the shaft;
an inner cover which is pulled over the shaft and the coil; and
an outer cover enclosing the inner cover, wherein the outer cover comprises a formed part for a balloon in its distal region for filling the volume between the outer cover and the inner cover with a liquid and/or gas to change the balloon in its size and/or to stiffen the balloon.

2. The endoscopic RF coil arrangement according to claim 1, wherein at least one connection is provided for introducing liquid and/or gas into the volume between the outer cover and the inner cover.

3. The endoscopic RF coil arrangement according to claim 1, wherein at least one connection is provided for evacuating the volume between the inner cover and the parts enclosed by the inner cover.

4. The endoscopic RF coil arrangement according to claim 1, wherein coils of different sizes can be connected with the inside tube, and an inner cover and an outer cover matching the coil size are each provided.

5. The endoscopic RF coil arrangement according to claim 1, wherein the inner cover is sealed by means of a seal and a flaring against the coil and the shaft and is fixed by means of a fastening element.

6. The endoscopic RF coil arrangement according claim 1, wherein the outer cover is sealed by means of a seal and a flaring against the coil and the shaft and is fixed by means of a fastening element.

7. The endoscopic RF coil arrangement according to claim 1, wherein at least one support is provided for receiving an additional instrument which is also completely enclosed by the outer cover.

8. The endoscopic RF coil arrangement according to claim 1, wherein the coil is connectable to an inner tube which is arranged in the interior of the shaft by means of a plug-and-socket connection, comprising a coupling and a plug.

9. The endoscopic RF coil arrangement according to claim 1, wherein the coil comprises a printed circuit board.

10. An endoscopic RF coil arrangement for a magnetic resonance tomograph, comprising:
a handle attached to a shaft;
a coil arranged distally to the shaft;
an outer cover which is pulled over the shaft and the coil; and
a support for an additional instrument, which is attached outside of the outer cover and penetrates the coil.

11. The endoscopic RF coil arrangement according to claim 10, wherein the outer cover is sealed by means of a seal and a flaring against the coil and the shaft and is fixed by means of a fastening element.

12. The endoscopic RF coil arrangement according to claim 10, wherein the coil is connectable to an inner tube which is arranged in the interior of the shaft by means of a plug-and-socket connection, comprising a coupling and a plug.

13. The endoscopic RF coil arrangement according to claim 10, wherein the coil comprises a printed circuit board.

14. An endoscopic instrument, comprising:
a handle attached to a shaft;
an instrument arranged distally to the shaft, having a head;
an outer cover which encloses the shaft and the head of the instrument; and
at least one support being provided for receiving an additional instrument which is also completely enclosed by the outer cover.

15. The endoscopic instrument according to claim 14, wherein the outer cover comprises in its distal region a formed part for a balloon, and a liquid and/or a gas can be introduced into the volume between the outer cover and the head of the instrument in order to change the balloon in its size and/or to stiffen the same.

16. The endoscopic instrument according to claim 14, wherein an inner cover is provided which encloses at least the head of the instrument.
